(19) United States
(12) Patent Application Publication
(10) Pub. No.: US 2024/0199578 A1
(43) Pub. Date: Jun. 20, 2024

(54) SOLID STATE FORMS OF ZAVEGEPANT AND PROCESS FOR PREPARATION THEREOF

(71) Applicant: TEVA CZECH INDUSTRIES S.R.O, Opava-Komarov (CZ)

(72) Inventors: Adéla Bártová, Ostrava-Michalkovice (CZ); Alexandr Jegorov, Dobrá Voda u Ceských Budejovic (CZ); Pavel Kolesa, Hlubocec (CZ); Zuzana Trcková, Ostrava (CZ)

(21) Appl. No.: 18/285,699

(22) PCT Filed: Apr. 8, 2022
(86) PCT No.: PCT/US2022/023943

§ 371 Date: Oct. 5, 2023

Related U.S. Application Data
(60) Provisional application No. 63/213,806, filed on Jun. 23, 2021.
(60) Provisional application No. 63/172,808, filed on Apr. 9, 2021.

(51) Int. Cl. C07D 401/14; A61K 31/496

(52) U.S. Cl. CPC C07D 401/14; A61K 31/496

(57) ABSTRACT

The present disclosure encompasses solid state forms of Zavegepant, in embodiments crystalline polymorphs of Zavegepant, processes for preparation thereof, and pharmaceutical compositions thereof.

### FIELD OF THE DISCLOSURE

The present disclosure encompasses solid state forms of Zavegepant, in embodiments crystalline polymorphs of Zavegepant, processes for preparation thereof, and pharmaceutical compositions thereof.

### BACKGROUND OF THE DISCLOSURE

Zavegepant, (R) N-(3-(7-methyl-1H-indazol-5-yl)-1-(4-(1-methylpiperidin-4-yl)piperazin-1-yl)-1-oxopropan-2-yl)-4-(2-oxo-1,2-dihydroquinolin-3-yl)piperidine-1-carboxamide, has the following chemical structure:

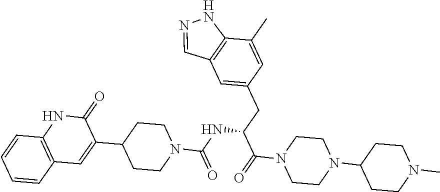 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]

Zavegepant is a third generation, high affinity, selective and structurally unique, small molecule CGRP receptor antagonist, and it is developed for acute treatment of migraine or COVID-19 respiratory infection.

The compound is described in International Publication No. WO2011/123232.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Zavegepant.

### SUMMARY OF THE DISCLOSURE

The present disclosure provides crystalline polymorphs of Zavegepant, processes for preparation thereof, and pharmaceutical compositions thereof. These crystalline polymorphs can be used to prepare other solid state forms of Zavegepant, Zavegepant salts and their solid state forms.

The present disclosure also provides uses of the said solid state forms of API in the preparation of other solid state forms of Zavegepant or salts thereof.

The present disclosure provides crystalline polymorphs of Zavegepant for use in medicine, including for the acute treatment of migraine, for the prevention of migraine, or for COVID-19 respiratory infection.

The present disclosure also encompasses the use of crystalline polymorphs of Zavegepant of the present disclosure for the preparation of pharmaceutical compositions and/or formulations, particularly pharmaceutical compositions or formulations for oral or intranasal administration, and especially for intranasal administration.

In another aspect, the present disclosure provides pharmaceutical compositions comprising crystalline polymorphs of Zavegepant according to the present disclosure. Pharmaceutical compositions according to any aspect of the present disclosure may include intranasal or oral dosage forms. Particularly, the pharmaceutical compositions may be an intranasal dosage form.

The present disclosure includes processes for preparing the above mentioned pharmaceutical compositions. The processes include combining any one or a combination of the crystalline polymorphs of Zavegepant with at least one pharmaceutically acceptable excipient. Particularly, the pharmaceutical compositions may comprise pharmaceutically acceptable excipient suitable for preparing an oral or intranasal dosage form, especially an intranasal dosage form.

The crystalline polymorph of Zavegepant as defined herein and the pharmaceutical compositions or formulations of the crystalline polymorph of Zavegepant may be used as medicaments, such as for the acute treatment of migraine, for the prevention of migraine, or COVID-19 respiratory infection.

The present disclosure also provides methods of treating migraine, preventing migraine or COVID-19 respiratory infection, by administering a therapeutically effective amount of any one or a combination of the crystalline polymorphs of Zavegepant of the present disclosure, or at least one of the above pharmaceutical compositions, to a subject suffering from migraine or COVID-19 respiratory infection, or otherwise in need of the treatment.

The present disclosure also provides uses of crystalline polymorphs of Zavegepant of the present disclosure, or at least one of the above pharmaceutical compositions, for the manufacture of medicaments for treating e.g., migraine or COVID-19 respiratory infection, or for preventing migraine. The medicament may be administered as an intranasal dosage form, or may be administered as an oral dosage form, particularly an intranasal dosage form.

According to any aspect or embodiment of the present disclosure, pharmaceutical compositions or formulations for the treatment of migraine or COVID-19 respiratory infection are preferably in the form of an intranasal dosage form. According to any aspect or embodiment of the present disclosure, pharmaceutical compositions or formulations for the prevention of migraine are preferably in the form of an oral dosage form.

### BRIEF DESCRIPTION OF THE DRAWINGS

FIG. 1a shows ¹³C solid state NMR spectrum of Form A1 of Zavegepant (full scan);

FIG. 1b shows ¹³C solid state NMR spectrum of Form A1 of Zavegepant (at the range of 230-100 ppm);

FIG. 1c shows ¹³C solid state NMR spectrum of Form A1 of Zavegepant (at the range of 100-0 ppm);

FIG. 2 shows a characteristic X-ray powder diffraction pattern (XRPD) of Zavegepant Form A1;

FIG. 3a shows ¹³C solid state NMR spectrum of Form A2 of Zavegepant (full scan);

FIG. 3b shows ¹³C solid state NMR spectrum of Form A2 of Zavegepant (at the range of 230-100 ppm);

FIG. 3c shows ¹³C solid state NMR spectrum of Form A2 of Zavegepant (at the range of 100-0 ppm);

FIG. 4 shows a characteristic X-ray powder diffraction pattern (XRPD) of Zavegepant Form A2;

FIG. 5a shows ¹³C solid state NMR spectrum of Form A3 of Zavegepant (full scan);

FIG. 5b shows ¹³C spectrum of Form A3 of Zavegepant (at the range of 230-100 ppm);

FIG. 5c shows ¹³C spectrum of Form A3 of Zavegepant (at the range of 100-0 ppm);

FIG. 6 shows a characteristic X-ray powder diffraction pattern (XRPD) of Zavegepant Form A3;

FIG. 7 shows a characteristic X-ray powder diffraction pattern (XRPD) of Zavegepant amorphous Form;

FIG. 8 shows a characteristic X-ray powder diffraction pattern (XRPD) of Zavegepant Form B;

FIG. 9a shows a characteristic solid state ¹³C NMR spectrum of Zavegepant Form B (at the range 200-0 ppm);

FIG. 9b shows a characteristic solid state ¹³C NMR spectrum of Zavegepant Form B (zoomed in at the range 200-100 ppm); and

FIG. 9c shows a characteristic solid state ¹³C NMR spectrum of Zavegepant Form B (zoomed in at the range 100-0 ppm).

### DETAILED DESCRIPTION OF THE DISCLOSURE

The present disclosure encompasses solid state forms of Zavegepant, including crystalline polymorphs of Zavegepant, processes for preparation thereof, and pharmaceutical compositions thereof.

Solid state properties of Zavegepant and crystalline polymorphs thereof can be influenced by controlling the conditions under which Zavegepant and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Zavegepant described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Zavegepant. In some embodiments of the disclosure, the described crystalline polymorph of Zavegepant may contain from about 1% to about 20% (w/w), from about 5% to about 20% (w/w), or from about 5% to about 10% (w/w) of one or more other crystalline polymorph of the same Zavegepant.

Depending on which other crystalline polymorphs a comparison is made, the crystalline polymorphs of Zavegepant of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Zavegepant referred to herein as being characterized by graphical data “as depicted in” or “as substantially depicted in” a Figure will thus be understood to include any crystal forms of Zavegepant characterized with the graphical data having such small variations, as are well known to the skilled person, in comparison with the Figure.

As used herein, and unless stated otherwise, the term “anhydrous” in relation to crystalline forms of Zavegepant, relates to a crystalline form of Zavegepant, which does not include any crystalline water (or other solvents) in a defined, stoichiometric amount within the crystal. Moreover, unless otherwise indicated, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Zavegepant of the present disclosure corresponds to a crystalline polymorph of Zavegepant that is physically separated from the reaction mixture in which it is formed.

As used herein, unless stated otherwise, the XRPD measurements are taken using copper Kα radiation wavelength 1.54187 Å. XRPD peaks reported herein are measured using Cuk α radiation, λ=1.54187 Å, typically at a temperature of 25±3° C.

As used herein, unless stated otherwise, ¹³C NMR reported herein are measured at 125 MHz at a magic angle spinning frequency ωᵣ/2π=11 kHz, preferably at a temperature of at 293 K±3° C.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

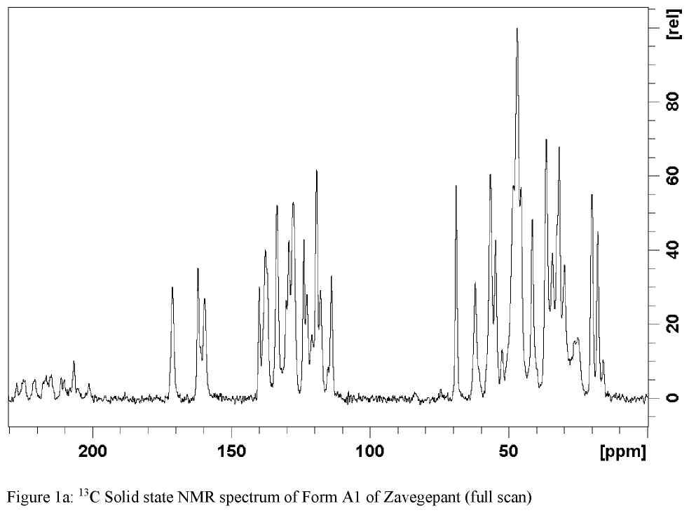

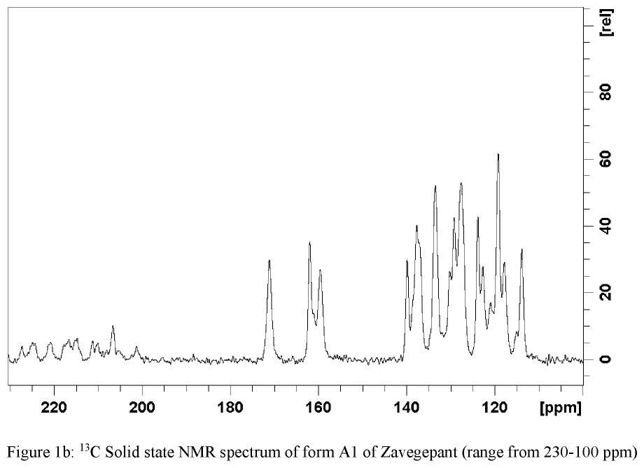

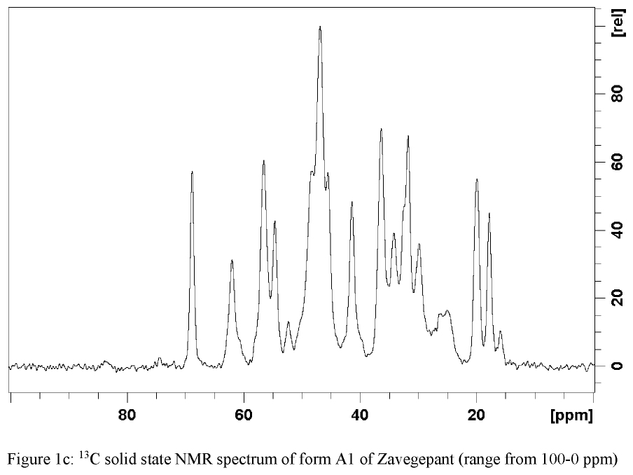

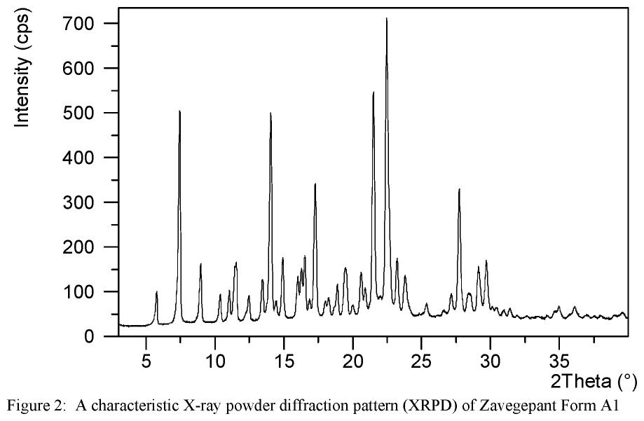

The present disclosure includes a crystalline polymorph of Zavegepant, designated A1. The crystalline Form A1 of Zavegepant may be characterized by data selected from one or more of the following: a solid state ¹³C NMR spectrum having peaks at the range of 0-100 ppm at 17.8, 36.3, 46.9, 56.5, 62.0±0.2 ppm; a solid state ¹³C NMR spectrum substantially as depicted in FIGS. 1a, 1b or 1c; an X-ray powder diffraction pattern substantially as depicted in FIG. 2; and combinations of these data.

In one embodiment of the present disclosure, crystalline Form A1 of Zavegepant is isolated.

Crystalline Form A1 of Zavegepant can be prepared by a process comprising slurrying Zavegepant, for example amorphous Zavegepant, in a solvent, such as iso-butanol, toluene, 1,4-dioxane or tetrahydrofuran (“THF”). Typically, this process can be performed at temperature of from about 50° C. to about 110° C. For example, slurrying Zavegepant in iso-butanol may be performed at temperature of: about 60° C. to about 100 ° C., about 70° C. to about 100° C., about 80° C. to about 100° C., or about 95° C. Slurrying Zavegepant in toluene may be performed at temperature of: about 50° C. to about 100° C., about 50° C. to about 80° C., about 50° C. to about 70° C., or about 60° C. Slurrying Zavegepant in 1,4-dioxane may be performed at temperature of: about 50° C. to about 110° C., about 50° C. to about 105° C., about 50° C. to about 100° C., or about 60 to about 95° C., or about 85° C. to about 95° C. Slurrying Zavegepant in THF may be performed at temperature of about 50° C. to about 100° C., about 50° C. to about 80° C., about 50° C. to about 70° C., or about 60° C. This step can be done for a period of time suitable to effect the conversion to Form A1 of Zavegepant, for example, for a period of from about 15 minutes to about 2 hours, or about 30 minutes.

The solid may be isolated by any suitable procedure, for example, centrifuge, filtration or decantation. Typically, the solid is filtered, for example by vacuum filtration.

The process may further comprise combining the Zavegepant Form A1 with at least one pharmaceutically acceptable excipient to produce a pharmaceutical composition or pharmaceutical formulation.

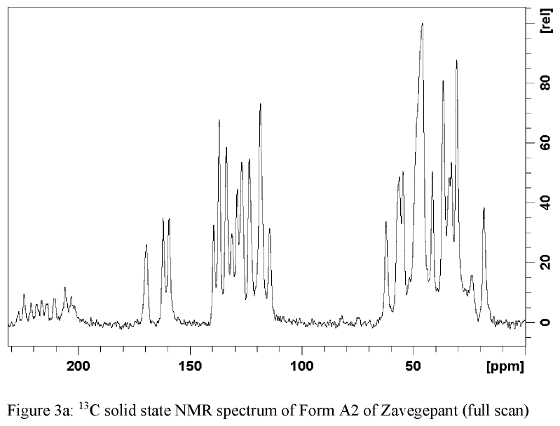

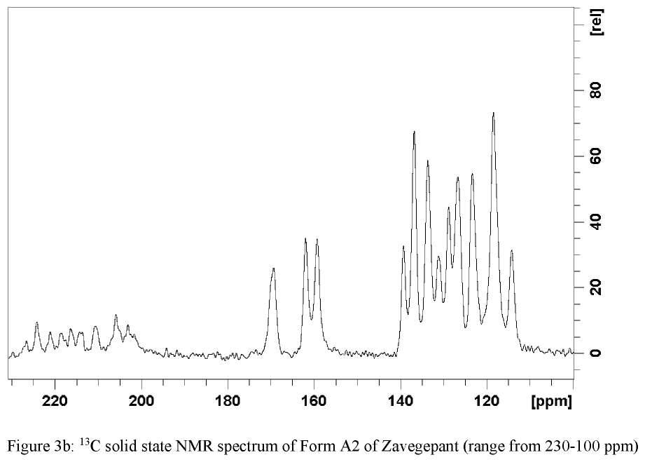

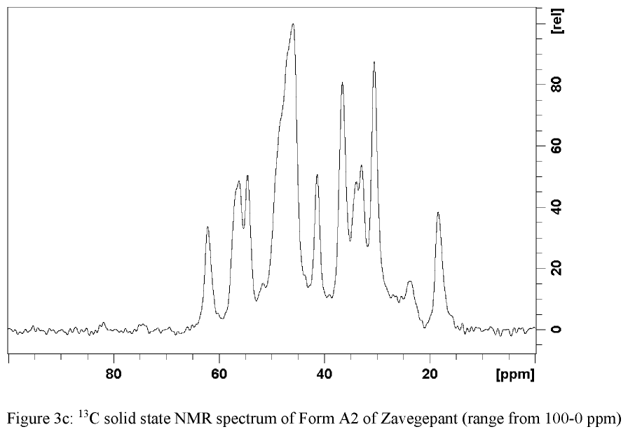

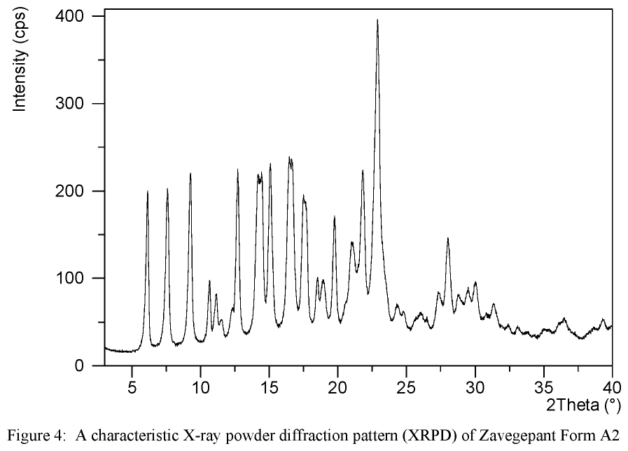

The present disclosure includes a crystalline polymorph of Zavegepant, designated A2. The crystalline Form A2 of Zavegepant may be characterized by data selected from one or more of the following: a solid state ¹³C NMR spectrum having peaks at the range of 0-100 ppm at 36.5, 41.3, 45.9, 56.1 and 62.1 ppm±0.2 ppm; a solid state ¹³C NMR spectrum substantially as depicted in FIG. 3a, 3b or 3c; an X-ray powder diffraction pattern substantially as depicted in FIG. 4; and combinations of these data.

Alternatively, crystalline Form A2 of Zavegepant may be characterized by the following unit cell data:

| Unit cell parameter | A2, LeBail 293 K |
|---|---|
| cell_length_a | 28.818 Å |
| cell_length_b | 12.711 Å |
| cell_length_c | 10.143 Å |
| cell_angle_alpha | 90° |
| cell_angle_beta | 90° |
| cell_angle_gamma | 90° |
| cell_volume | 3715.5 Å³ |
| symmetry_cell_setting | orthorhombic |
| Symmetry space group name | P 2₁2₁2₁ |

as determined at a temperature of about 293° K.

In one embodiment of the present disclosure, crystalline Form A2 of Zavegepant is isolated.

Crystalline Form A2 of Zavegepant may be characterized by each of the above characteristics alone or by all possible combinations, e.g., a ¹³C NMR spectrum having peaks at the range of 0-100 ppm at 36.5, 41.3, 45.9, 56.1 and 62.1 ppm±0.2 ppm; an XRPD pattern as depicted in FIG. 4; or the unit cell data described above; and combinations thereof.

Crystalline Form A2 of Zavegepant according to any embodiment or aspect of the present disclosure may optionally contain water, preferably in an amount of: about 0.6 to 5% of water, about 0.8 to about 4% of water, about 1.0 to about 3.0% of water, or about 1.2 to about 2.4% of water, by weight, as measured by TGA. Crystalline Form A2 of Zavegepant can be prepared by a process comprising slurrying Zavegepant, for example amorphous Zavegepant, in a solvent, such as ethyl acetate, methyl ethyl ketone (“MEK”) or acetone. Typically, this process can be performed at temperature of from about 40° C. to about 80° C. For example, slurrying Zavegepant in ethyl acetate may be performed at temperature of: about 50° C. to about 80° C., about 60° C. to about 75° C., about 65° C. to about 75° C., or about 70° C. Slurrying Zavegepant in MEK may be performed at temperature of: about 40° C. to about 75° C., about 40° C. to about 60° C., about 45° C. to about 55° C., or about 50° C. Slurrying Zavegepant in acetone may be performed at temperature of: about 40° C. to about 70° C., about 40° C. to about 55° C., about 45° C. to about 50° C., or about 45° C. This step can be done for a period of time suitable to effect the conversion to Form A2 of Zavegepant, for example, for a period of from about 15 minutes to about 2 hours, or about 30 minutes.

The solid may be isolated by any suitable procedure, for example, centrifuge, filtration or decantation. Typically, the solid is filtered, for example by vacuum filtration. The solid can then be further dried, for example at temperature of from about 70° C. to about 90° C., typically about 80° C.; it can be done for a suitable time, for example from about 3 hours to about 6 hours, typically about 5 hours. The drying can be done under nitrogen.

The process may further comprise combining the Zavegepant Form A2 with at least one pharmaceutically acceptable excipient to produce a pharmaceutical composition or pharmaceutical formulation.

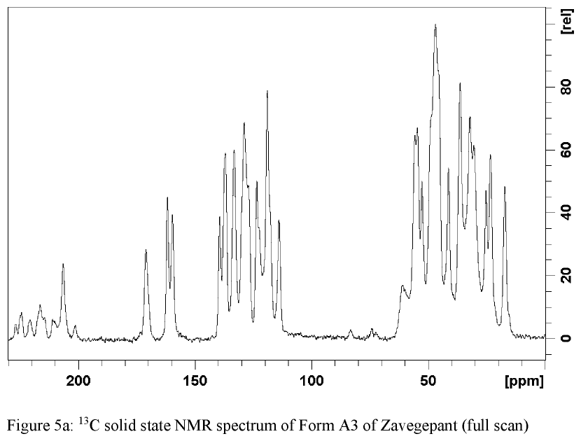

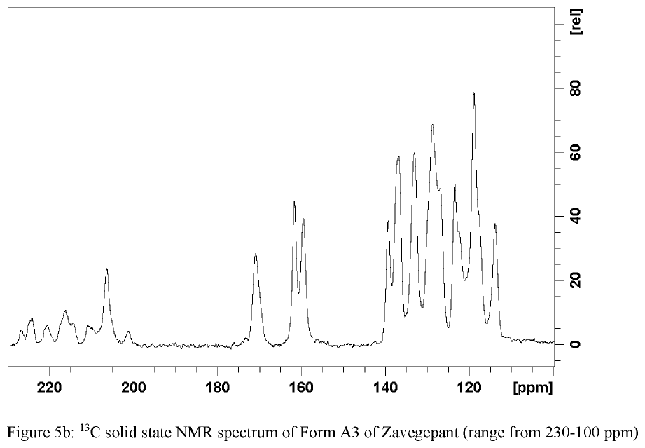

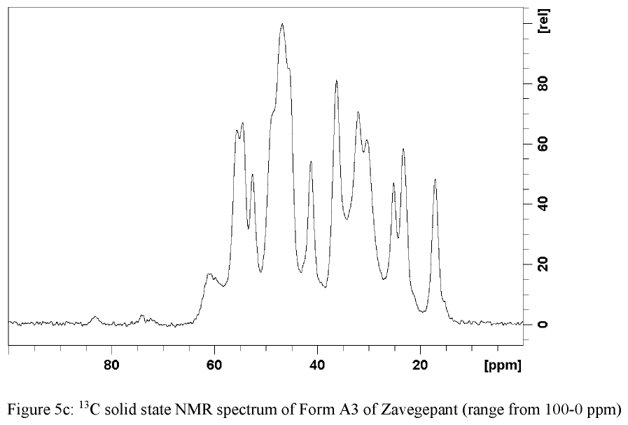

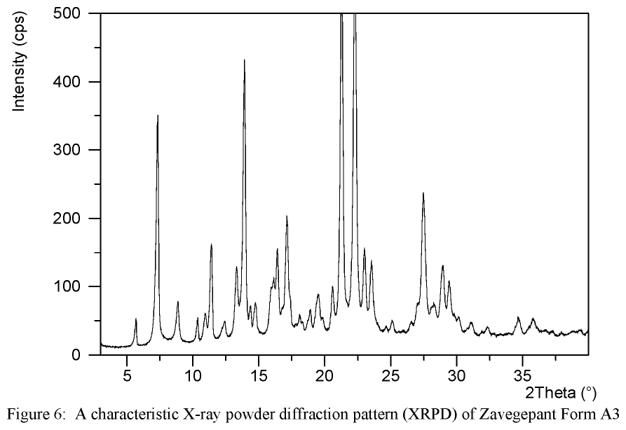

The present disclosure includes a crystalline polymorph of Zavegepant, designated A3. The crystalline Form A3 of Zavegepant may be characterized by data selected from one or more of the following: a solid state ¹³C NMR spectrum having peaks at the range of 0-100 ppm at 17.1, 25.1, 32.1, 41.2, 46.8±0.2 ppm; a solid state ¹³C NMR spectrum substantially as depicted in FIG. 5a, 5b or 5c; an X-ray powder diffraction pattern substantially as depicted in FIG. 6; and combinations of these data.

In one embodiment of the present disclosure, crystalline Form A3 of Zavegepant is isolated.

Crystalline Form A3 of Zavegepant can be prepared by a process comprising slurrying Zavegepant, for example amorphous Zavegepant, in a solvent, such as isoamyl alcohol or methyl iso-butyl ketone (“MIBK”). Typically, this process can be performed at temperature of: about 70° C. to about 120° C., about 80° C. to about 110° C., about 90° C. to about 100° C., or about 95° C. This step can be done for a period of time suitable to effect the conversion to Form A3 of Zavegepant, for example, for this step can be done for a period of from about 15 minutes to about 2 hours, or about 30 minutes.

The solid may be isolated by any suitable procedure, for example, centrifuge, filtration or decantation. Typically, the solid is filtered, for example by vacuum filtration.

The process may further comprise combining the Zavegepant Form A3 with at least one pharmaceutically acceptable excipient to produce a pharmaceutical composition or pharmaceutical formulation.

The present disclosure includes a crystalline polymorph of Zavegepant, designated Form B. Crystalline Form B may be described with reference to an XRPD pattern having characteristic peaks at 3.9, 12.4 and 13.1 degrees 2-theta±0.2 degrees 2-thetaCrystalline Form B may be described with reference to an XRPD pattern having characteristic peaks at 3.9, 12.4 and 13.1 degrees 2-theta±0.2 degrees 2-theta, and one or two additional peaks at 7.7 and 17.5 degrees 2-theta±0.2 degrees 2-theta. Alternatively, crystalline Form B may be described as above, and further having any one, two, three, or four additional peaks selected from 15.4, 18.4, 19.2 and 24.7 degrees 2-theta±0.2 degrees 2-theta. Crystalline Form B may be defined according to any of the characteristic XRPD peaks as described above, and additionally having an absence of peaks: between 4.4 to 6.8 degrees 2-theta±0.2 degrees 2-theta; or preferably between 5.0 to 6.8 degrees 2-theta±0.2 degrees 2-theta.

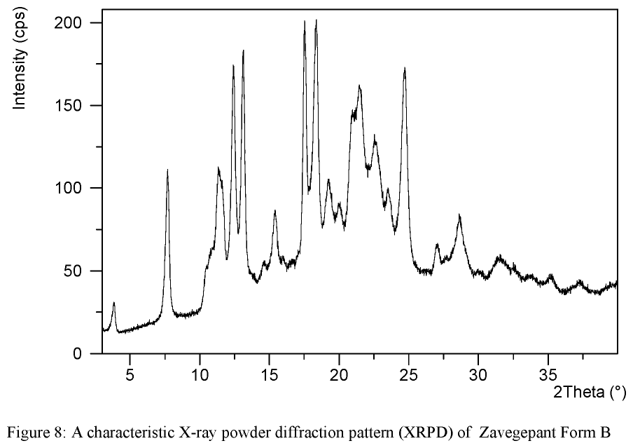

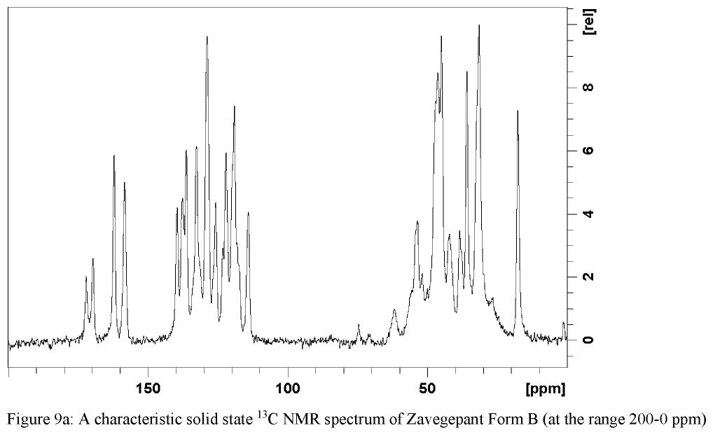

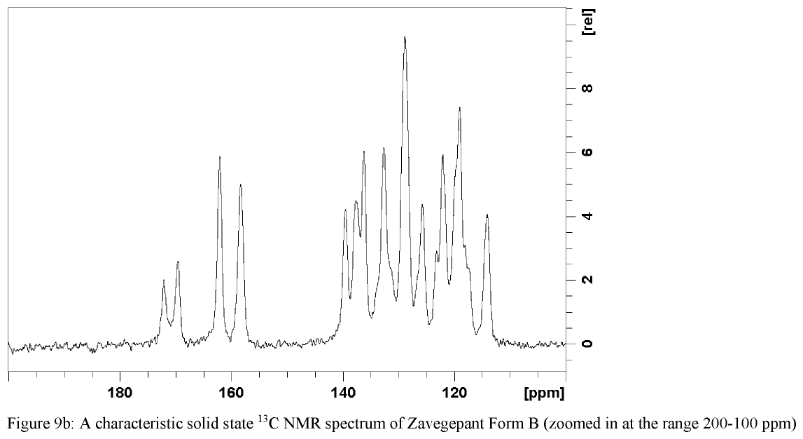

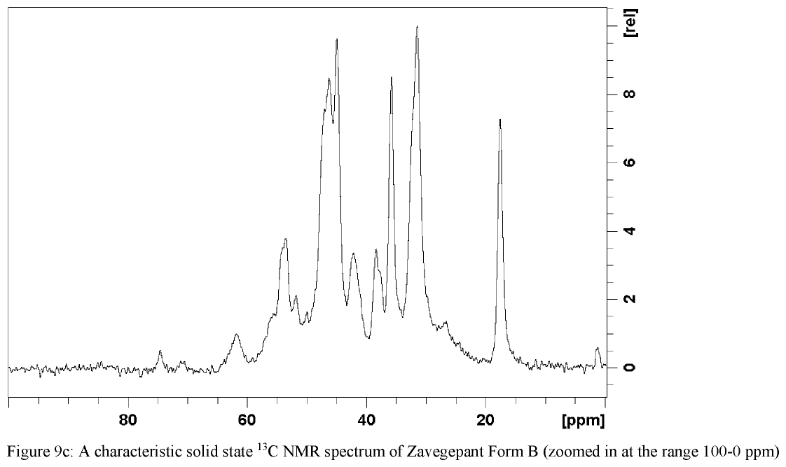

Alternatively, the crystalline Form B of Zavegepant may be characterized by data selected from one or more of the following: an X-ray powder diffraction pattern substantially as depicted in FIG. 8; an X-ray powder diffraction pattern having peaks at 3.9, 7.7, 12.4, 13.1 and 17.5 degrees 2-theta±0.2 degrees 2-theta : a solid state ¹³C NMR spectrum having peaks at the range of 0-100 ppm at 17.6, 31.5, 35.8, 45.0 and 46.3±0.2 ppm; a solid state ¹³C NMR spectrum substantially as depicted in either FIG. 9a, 9b or 9c; and combinations of these data. Crystalline Form B may be defined by an X-ray powder diffraction pattern having peaks at 3.9, 7.7, 12.4, 13.1 and 17.5 degrees 2-theta±0.2 degrees 2-theta, and additionally having an absence of peaks between 4.4 to 6.8 degrees 2-theta±0.2 degrees 2-theta; or preferably between 5.0 to 6.8 degrees 2-theta±0.2 degrees 2-theta.

Crystalline Form B of Zavegepant may be further characterized by an X-ray powder diffraction pattern having peaks at 3.9, 7.7, 12.4, 13.1 and 17.5 degrees 2-theta±0.2 degrees 2-theta, and also having any one, two, three, or four additional peaks selected from 15.4, 18.4, 19.2 and 24.7 degrees 2-theta±0.2 degrees 2-theta. Crystalline Form B of Zavegepant may be further characterized by an X-ray powder diffraction pattern having peaks at 3.9, 7.7, 12.4, 13.1 and 17.5 degrees 2-theta±0.2 degrees 2-theta, and also having any one, two, three, or four additional peaks selected from 15.4, 18.4, 19.2 and 24.7 degrees 2-theta±0.2 degrees 2-theta; and additionally having an absence of peaks between 4.4 to 6.8 degrees 2-theta±0.2 degrees 2-theta; or preferably between 5.0 to 6.8 degrees 2-theta±0.2 degrees 2-theta.

Alternatively, crystalline Form B of Zavegepant may be characterized by an XRPD having peaks at: 3.9, 7.7, 11.7, 12.4, 13.1, 14.6, 15.4, 17.5, 18.4, 19.2, 20.0, 20.9, 21.4, 22.6, 23.5, 24.7, 27.1 and 28.7 degrees 2-theta±0.2 degrees 2-theta; and optionally having an absence of peaks between 4.4 to 6.8 degrees 2-theta±0.2 degrees 2-theta; or an absence of peaks between 5.0 to 6.8 degrees 2-theta±0.2 degrees 2-theta.

Alternatively, crystalline Form B of Zavegepant according to any aspect or embodiment of the present disclosure may be characterized by an X-ray powder diffraction pattern having peaks as set out in Table 1 below:

TABLE 1
Pos. [°2θ ± 0.2 degrees 2-theta]

3.9
7.7
11.7
12.4
13.1
14.6
15.4
17.5
18.4
19.2
20.0
20.9
21.4
22.6
23.5
24.7
27.1
28.7

In one embodiment of the present disclosure, crystalline Form B of Zavegepant is isolated.

According to any aspect or embodiment of the present disclosure, crystalline Form B of Zavegepant is preferably isolated.

According to any aspect or embodiment of the present disclosure, crystalline Form B of Zavegepant is anhydrous (i.e. a non-hydrated form, or an anhydrate). In particular, crystalline Form B of Zavegepant according to any aspect or embodiment of the present disclosure does not include any crystalline water (i.e. water bound within the crystal). Crystalline Form B may, nevertheless, optionally contain water (i.e. free, or unbound water, which is not part of the crystal), optionally in an amount of: about 0.6 to 10% of water, about 0.8 to about 5% of water, about 1.0 to about 3.0% of water, or about 1.3 to about 2.6% of water, by weight as measured by TGA.

Crystalline Form B of Zavegepant may be characterized by each of the above characteristics alone/or by all possible combinations, e.g., an XRPD pattern having peaks at 3.9, 7.7, 12.4, 13.1 and 17.5 degrees 2-theta±0.2 degrees 2-theta; an XRPD pattern as depicted in FIG. 8, and combinations thereof.

Crystalline Form B of Zavegepant may have advantageous properties, as discussed above. For example, crystalline Form B is particularly stable to polymorphic conversion when heated, during grinding and/or high relative humidity.

Crystalline Form B of Zavegepant can be prepared by a process comprising slurrying Zavegepant in acetonitrile. The Zavegepant may be an amorphous form. Typically, this process can be performed at temperature of: about 40° C. to about 85° C., about 40° C. to about 70° C., about 40° C. to about 60° C., or about 45° C. to about 55° C., typically about 50° C. This step can be done for a period of time suitable to effect the conversion to Form B of Zavegepant, for example, for a few days, such as 3 days or 4 days.

The solid may be isolated by any suitable procedure, for example, centrifuge, filtration or decantation. Preferably, the Form B may be isolated by filtration, for example by vacuum filtration.

The solid can then be dried on the filter, or may further dried, for example under vacuum at about room temperature and for sufficient time, such as 15 minutes to about 1 hour. Alternatively, drying can be done at temperature of from about 50° C.; for example for a period of from about 3 hours to about 6 hours, typically about 5 hours. Such drying can be done under nitrogen.

The process may further comprise combining the Zavegepant Form B with at least one pharmaceutically acceptable excipient to produce a pharmaceutical composition or pharmaceutical formulation.

In any aspect or embodiment of the present disclosure, any of the solid state forms of Zavegepant described herein may be polymorphically pure, or may be substantially free of any other solid state forms of Zavegepant. In any aspect or embodiment of the present disclosure, any of the solid state forms of Zavegepant disclosed herein, may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0% of any other solid state forms of Zavegepant, preferably as measured by XRPD. Thus, any of the disclosed crystalline forms of Zavegepant described herein may be substantially free of any other solid state forms of Zavegepant, and may contain greater than about 80% (w/w), greater than about 90% (w/w), greater than about 95% (w/w), greater than about 98% (w/w), greater than about 99% (w/w), or about 100% of the subject solid state form of the Zavegepant.

The above crystalline polymorphs can be used to prepare other crystalline polymorphs of Zavegepant, Zavegepant salts and their solid state forms. Solid state forms may crystalline polymorphs, co-crystals and complexes of Zavegepant or of Zavegepant salt.

The present disclosure encompasses a process for preparing other solid state forms of Zavegepant and their solid state forms thereof. The process includes preparing any one of the crystalline polymorphs of Zavegepant by the processes of the present disclosure. The process may further comprise converting said crystalline polymorph of Zavegepant to other crystalline polymorph of Zavegepant or Zavegepant salt.

The present disclosure provides the above described crystalline polymorphs of Zavegepant for use in the preparation of pharmaceutical compositions comprising Zavegepant and/or crystalline polymorphs thereof.

The present disclosure also encompasses the use of crystalline polymorphs of Zavegepant of the present disclosure for the preparation of pharmaceutical compositions of crystalline polymorph Zavegepant and/or crystalline polymorphs thereof. Particularly, the pharmaceutical compositions may be used for intranasal or oral administration, and more particularly for intranasal administration.

The present disclosure includes processes for preparing the above mentioned pharmaceutical compositions. The processes include combining any one or a combination of the crystalline polymorphs of Zavegepant of the present disclosure with at least one pharmaceutically acceptable excipient. Particularly, the pharmaceutical compositions may comprise pharmaceutically acceptable excipient suitable for making formulation of intranasal or oral administration, and more particularly for intranasal administration. Pharmaceutical combinations or formulations of the present disclosure contain any one or a combination of the solid state forms of Zavegepant of the present disclosure. In addition to the active ingredient, the pharmaceutical formulations of the present disclosure can contain one or more excipients. Excipients are added to the formulation for a variety of purposes. For example, excipients may be added to assist in formation of formulation suitable for intranasal administration.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Zavegepant and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, intranasal and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Zavegepant can be administered. For example, it can be administrated intranasally or orally. Zavegepant may be formulated for administration to a mammal, in embodiments to a human, by injection. Zavegepant can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Zavegepant and the pharmaceutical compositions and/or formulations of Zavegepant of the present disclosure can be used as medicaments, in embodiments in the treatment of migraine or COVID-19 respiratory infection. The medicament may preferably be administrated in intranasal or oral form, and more preferably in intranasal form.

The present disclosure also provides methods of treating migraine or COVID-19 respiratory infection by administering a therapeutically effective amount of any one or a combination of the crystalline polymorphs of Zavegepant of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-ray Diffraction (“XRPD”) method

Powder X-ray Diffraction was performed on an X-Ray powder diffractometer PanAlytical X′pert Pro; CuKα radiation (λ=1.54187 Å); X′Celerator detector with active length 2.122 degrees 2-theta; laboratory temperature 25±3° C.; zero background sample holders. Prior to analysis, the samples were gently ground using a mortar and pestle to obtain a fine powder. The ground sample was adjusted into a cavity of the sample holder and the surface of the sample was smoothed using a cover glass.

Measurement parameters:

| Scan range: | 3-40 degrees 2-theta | |
|---|---|---|
| Scan mode: | continuous | |
| Step size: | 0.0167 | degrees |
| Step size: | 42 | s |
| Sample spin: | 60 | rpm |

| | |
|---|---|
| Sample holder: | zero background silicon plate |

### ¹³C NMR Method

All ¹³C CP/MAS NMR spectra were measured at 125 MHz using Bruker Avance 500 WB/US NMR spectrometer (Karlsruhe, Germany, 2003) at magic angle spinning (MAS) frequency ωᵣ/2π=11 kHz. In all cases finely powdered samples were placed into the 4 mm ZrO₂ rotors and standard CPMAS pulse program was used. During acquisition of the data a high-power dipolar decoupling TPPM (two-pulse phase-modulated) was applied. The number of scans was 5120 for forms A1 and A2, 15 360 for forms A3 and 2048 for form B; repetition delay was 3 s for forms A1, A3 and B, 2 s for form A2. Taking into account frictional heating of the samples during fast rotation all NMR experiments were performed at 293° K (precise temperature calibration was performed).

### X-Ray Crystal Structure Determination—Form A2

Indexation was calculated from regular XRPD scan indexation was calculated:
Program: Highscore
Method: LeBail fit

### EXAMPLES

### Preparation of Starting Materials

Zavegepant can be prepared according to methods known from the literature, for example U.S. Pat. No. 8,481,546

### Example 1: Preparation of Zavegepant Form A1

Zavegepant (amorphous, 80 mg) was slurried at temperature of about 95° C. for about 30 minutes in iso-butanol (1 ml). Sample was filtered and analyzed by XRPD. Zavegepant Form A1 was obtained. An XRPD pattern is shown in FIG. 2.

### Example 2: Preparation of Zavegepant Form A1

Zavegepant (amorphous, 80 mg) was slurried at temperature of about 60° C. for about 30 minutes in toluene (1 ml). Sample was filtered and analyzed by XRPD. Zavegepant Form A1 was obtained.

### Example 3: Preparation of Zavegepant Form A1 Procedure A:

Zavegepant (amorphous, 80 mg) was slurried at temperature of about 60° C. for about 30 minutes in 1,4-dioxane (1 ml). Sample was filtered and analyzed by XRPD. Zavegepant Form A1 was obtained.

### Example 3: Preparation of Zavegepant Form A1 Procedure B

Zavegepant (amorphous, 80 mg) was slurried at temperature of about 95° C. for about 30 minutes in 1,4-dioxane (1 ml). Sample was filtered and analyzed by XRPD. Zavegepant Form A1 was obtained.

### Example 4: Preparation of Zavegepant Form A1

Zavegepant (amorphous, 80 mg) was slurried at temperature of about 60° C for about 30 minutes in THF (1 ml). Sample was filtered and analyzed by XRPD. Zavegepant Form A1 was obtained.

### Example 5: Preparation of Zavegepant Form A2

Zavegepant (amorphous, 80 mg) was slurried at temperature of about 70° C. for about 30 minutes in ethyl acetate (1 ml). Sample was filtered and analyzed by XRPD. Zavegepant Form A2 was obtained. An XRPD pattern is shown in FIG. 4.

### Example 6: Preparation of Zavegepant Form A2

Zavegepant (amorphous, 80 mg) was slurried at temperature of about 50° C. for about 30 minutes in methyl ethyl ketone (1 ml). Sample was filtered and analyzed by XRPD. Zavegepant Form A2 was obtained.

### Example 7: Preparation of Zavegepant Form A3

Zavegepant (amorphous, 80 mg) was slurried at temperature of about 95° C. for about 30 minutes in isoamyl alcohol (1 ml). Sample was filtered and analyzed by XRPD. Zavegepant Form A3 was obtained. An XRPD pattern is shown in FIG. 6.

### Example 8: Preparation of Zavegepant Form A3

Zavegepant (amorphous, 80 mg) was slurried at temperature of about 95° C. for about 30 minutes in methyl iso-butyl ketone. Sample was filtered and analyzed by XRPD. Zavegepant Form A3 was obtained.

### Example 9: Preparation of Zavegepant Amorphous Form

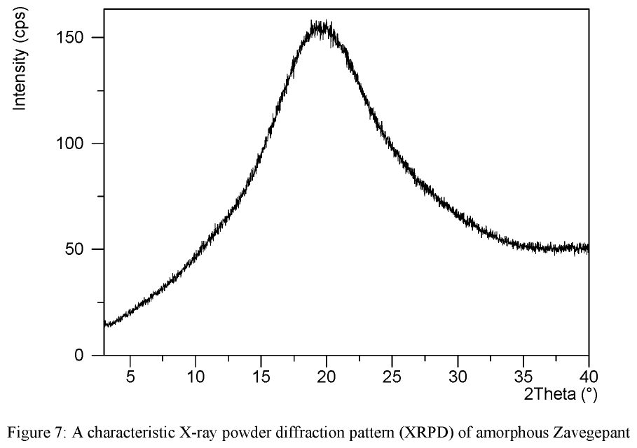

Zavegepant hydrochloride (2 grams) was dissolved in a mixture of water (35 ml) and methanol (5 ml). Dichloromethane was added (25 ml). Potassium carbonate was added dropwise to the clear solution until pH reached 11. Zavegepant was precipitated. Dichloromethane was added until dissolution of Zavegepant. The layers were separated and organic phase was evaporated to dryness on vacuum evaporator. The sample was analyzed by XRPD. Amorphous Zavegepant base was obtained. An XRPD pattern is shown in FIG. 7.

### Example 10: Preparation of Zavegepant Form B

Zavegepant (amorphous, 300 mg) was suspended in acetonitrile (3.5 ml) and the suspension was stirred for period of about 3 days at temperature of about 50° C. The obtained solid was filtered, dried under vacuum for period of about 15 minutes at room temperature and analyzed by XRPD. Zavegepant Form B was obtained. An XRPD pattern is shown in FIG. 8.

### Example 11: Preparation of Zavegepant Form B

Zavegepant (amorphous, 1 gram) was suspended in acetonitrile (20 ml) and the suspension was stirred for period of 3 days at temperature of about 50° C. The obtained solid was filtered , dried at room temperature for period of 2 hours and dried under stream of nitrogen for period of 5 hours at temperature of about 50° C. and analyzed by XRPD. Zavegepant Form B was obtained.

### Example 12: Preparation of Zavegepant Form A2

Zavegepant (3.2 grams; amorphous) was slurried at 45° C. for 1 hour in acetone (40 ml). The crystalline phase was separated by filtration and dried under vacuum for 30 minutes. The obtained solid was dried under a stream of nitrogen for 5 hours at 80° C. Form A2 was obtained in a yield of 94%. A sample was taken and analyzed for crystal structure determination.

### Example 13: Preparation of Zavegepant Form B

Zavegepant (3.1 grams; amorphous) was slurried in acetonitrile (60 ml) for 4 days at 50° C. The crystalline phase was separated by filtration and dried under vacuum for 1 hour. Form B was obtained in the yield of 96%. A sample was taken and analyzed by ssNMR.

1. Crystalline Form B of Zavegepant characterized by data selected from one or more of the following:
a) an X-ray powder diffraction pattern substantially as depicted in FIG. 8;
b) an X-ray powder diffraction pattern having peaks at 3.9, 12.4 and 13.1 degrees 2-theta±0.2 degrees 2-theta;
c) an X-ray powder diffraction pattern having peaks at 3.9, 7.7, 12.4, 13.1 and 17.5 degrees 2-theta±0.2 degrees 2-theta;
d) a solid state ¹³C NMR spectrum having peaks at the range of 0-100 ppm at 17.6, 31.5, 35.8, 45.0 and 46.3±0.2 ppm;
e) a solid state ¹³C NMR substantially as depicted in either FIG. 9a, 9b or 9c; and
f) any combination of (a)-(e).

2. Crystalline Form B of Zavegepant according to claim 1 which is characterised by an X-ray powder diffraction pattern having peaks at 3.9, 12.4 and 13.1 degrees 2-theta±0.2 degrees 2-theta and further having one or two additional peaks at 7.7 and 17.5 degrees 2-theta±0.2 degrees 2-theta.

3. Crystalline Form B of Zavegepant according to claim 2 which is further characterized by an XRPD pattern having any one, two, three, or four additional peaks selected from 15.4, 18.4, 19.2 and 24.7 degrees 2-theta±0.2 degrees 2-theta.

4. Crystalline Form B of Zavegepant according to claim 1, characterized by: an X-ray powder diffraction pattern having peaks at 3.9, 7.7, 12.4, 13.1 and 17.5 degrees 2-theta±0.2 degrees 2-theta, and also having any one, two, three or four additional peaks selected from 15.4, 18.4, 19.2 and 24.7 degrees 2-theta±0.2 degrees 2-theta.

5. Crystalline Form B of Zavegepant according to claim 1, which is further characterised by an X-ray powder diffraction pattern having an absence of peaks between 4.4 to 6.8 degrees 2-theta±0.2 degrees 2-theta; or an absence of peaks between 5.0 to 6.8 degrees 2-theta±0.2 degrees 2-theta.

6. Crystalline Form B of Zavegepant according to claim 1, which is an anhydrous (i.e. non-hydrate) form.

7. Crystalline Form B of Zavegepant according to claim 1, containing from about 0.6 to about 10% of water, by weight.

8. Crystalline Form B of Zavegepant according to claim 1, which contains no more than about 20% of any other crystalline forms of Zavegepant.

9. Crystalline Form B of Zavegepant according to claim 1, which contains no more than about 20% of amorphous Zavegepant.

10. A pharmaceutical composition comprising a crystalline form according to claim 1.

11. A pharmaceutical formulation comprising a crystalline form according to claim 1, and at least one pharmaceutically acceptable excipient.

12. A pharmaceutical formulation according to claim 11, wherein the formulation is an intranasal formulation.

13. A process for preparing a pharmaceutical formulation comprising combining a crystalline form according to claim 1 with at least one pharmaceutically acceptable excipient.

14. (canceled)

15. (canceled)

16. (canceled)

17. A method of treating cancer, migraines, or COVID-19 respiratory infection, comprising administering a therapeutically effective amount of a crystalline form according to claim 1 to a subject in need of the treatment.

18. The method according to claim 17, wherein the treatment is administrated intranasally.)

19. (canceled)

20. (canceled)

21. A process for preparing solid state form of Zavegepant, Zavegepant salt and their crystalline polymorph, co-crystals or complex thereof, comprising preparing a crystalline form according to claim 1, and converting it to another solid state form of Zavegepant, Zavegepant salt and their crystalline polymorph, co-crystals or complex thereof.
